(19) United States
(12) Patent Application Publication
(10) Pub. No.: US 2007/0150042 A1
(43) Pub. Date: Jun. 28, 2007

(54) STENTS WITH BEVELED ENDS AND METHODS OF USE THEREOF

(76) Inventor: Malur R. Balaji, Pittsford, NY (US)

Correspondence Address: BURNS & LEVINSON, LLP, 125 SUMMER STREET, BOSTON, MA, 02110, US

(21) Appl. No.: 11/566,393

(22) Filed: Dec. 4, 2006

Related U.S. Application Data
(60) Provisional application No. 60/742,316, filed on Dec. 5, 2005.

(51) Int. Cl. A61F 2/06

(52) U.S. Cl. 623/1.11; 623/1.13; 623/1.15

(57) ABSTRACT

Apparatus and methods for endovascular procedures for stenting an anatomical lumen.

### CROSS REFERENCE TO RELATED APPLICATIONS

This application claims priority of U.S. Provisional Application 60/742,316, entitled STENTS WITH BEVELED ENDS AND METHODS OF THE USE THEREOF, also filed on Dec. 5, 2005, which is hereby incorporated by reference herein.

### BACKGROUND

These teachings relate to the apparatus and methods for endorvascular procedures for stenting an anatomical lumen.

Stents are generally tubular-shaped devices which function to hold open a segment of a blood vessel or other anatomical lumen. They are particularly suitable for use to support and hold back a dissected arterial lining which can occlude the fluid passageway therethrough.

Stenting is the permanent placement of a small, latticed tube inside an anatomical lumen to provide structural support and to keep the lumen (hollow channel) open to maintain blood flow. The stenting procedure involves passing a collapsed stent into the artery to the site that requires support. The lattices of the stent are then allowed to expand, increasing the diameter of the stent. The expanded stent is then left permanently in place in the vessel.

Various means have been described to deliver and implant stents. One method frequently described for delivering a stent to a desired intraluminal location includes mounting the expandable stent on an expandable member, such as a balloon, provided on the distal end of an intravascular catheter, advancing the catheter to the desired location within the patient's body lumen, inflating the balloon on the catheter to expand the stent into a permanent expanded condition and then deflating the balloon and removing the catheter.

The current design of vascular stents is a tube whose wall is constructed of an expandable, structural, open-lattice made of a material such as nickel titanium (NiTinol), stainless steel, or other materials. In conventional designs, the ends of the stent are cut substantially perpendicular to the long axis of the stent.

The characteristics of existing stent designs make the insertion of a catheter into the lumen of a stented vessel difficult. The perpendicular end of a stent creates an abrupt transition between the vessel and the stent. A catheter inserted into the artery will encounter the pointed tips of the stent lattices at the same position along the length of the artery. A common practice for cannulation (the insertion of a cannula or tube such as a stent into a vessel of the body) is to use an angled catheter that can be rotated to move the distal end of the catheter away from obstructions, including the tips of the lattices located at the proximal end of the stent. However, the perpendicular cut of stent increases the likelihood that the distal end of the catheter will collide with the proximal end of one or more lattices—even when the catheter is rotated as shown in FIG. 3. That is, the perpendicular cut of current stents creates an obstacle that challenges the current practices of cannulation. Great difficulty may be encountered when attempting to pass a catheter through the site because the catheter may be unable to turn within the tight radius, or the end of the catheter may become caught on the proximal edge of the stent, or the end of the catheter may become caught between the outer surface of the stent and the inner surface of the artery.

The characteristics of existing stent designs used in stenting branch arteries can result on narrowing (bottle necking) of the mouth of the branch arteries. The current method for stenting branch arteries is to insert the stent such that its entire length is within the branch. That is, the proximal end of the stent is inside the branch artery, downstream of the mouth. As a result, the mouth of the artery, which is not supported by the stent, may decrease in diameter (see FIG. 4). This condition is sometimes referred to as bottle necking because the diameter at the mouth of the branch artery becomes smaller like the neck of a bottle. This condition can impede the flow of blood into the branch artery. It is not desirable to position a stent such that its proximal end extends into the main artery. While this position would provide greater support to the mouth of the branch artery, the proximal end of the stent would impede blood flow in the main artery. Therefore, there is presently no simple endovascular procedure to counteract bottlenecking.

Conventional stenting practice does not include special stent configurations to accommodate the curvature of tortuous arteries. Stents used in tortuous arteries have a tubular shape and perpendicular ends similar to stents used in other vessels. A tortuous artery will conform to the straight tubular shape of the stent. However, beyond the distal end of the stent, the artery will tend to turn or kink at an acute angle, as shown in FIG. 5. This angle can be so acute that it partially restricts the blood flow or causes turbulence that can impede the flow of blood, cause damage to cells in the blood as they pass through the turbulence, and contribute to the accumulation of plaque near the site.

Stents are also used in stent grafts. Aneurysms occur in blood vessels in locations where, due to age, disease or genetic predisposition, the blood vessel strength or resiliency is insufficient to enable the blood vessel wall to retain its shape as blood flows therethrough, resulting in a ballooning or stretching of the blood vessel at the limited strength/resiliency location to thereby form an aneurysmal sac. If the aneurysm is left untreated, the blood vessel wall may continue to expand, to the point where the remaining strength of the blood vessel wall is below that necessary to prevent rupture, and the blood vessel will fail at the aneurysm location, often with fatal result.

To prevent rupture of an aortic aneurysm, a stent graft of a tubular construction is introduced into the blood vessel, such as from a remote location through a catheter introduced into a major blood vessel in the leg, in one instance, and pushed through the blood vessel to the aneurysm location. The stent graft is deployed and secured in a location within the blood vessel such that the stent graft spans the aneurysmal sac. The outer surface of the stent graft, at its opposed ends, is sealed to the interior wall of the blood vessel (aorta) at a location where the blood vessel wall has not suffered a loss of strength or resiliency, such that blood flowing through the vessel is channeled through the hollow interior of the stent graft, and thus reduces, if not eliminates, the stress on the blood vessel wall at the aneurysmal sac location. Therefore, the risk of rupture of the blood vessel wall at the aneurysmal location is significantly reduced, if not eliminated, and blood can continue to flow through to the downstream blood vessels without interruption.

In one embodiment, stent grafts are typically configured by separately forming the graft and the stent, and then attaching the graft to the stent. The graft provides a tubular pathway for blood to flow past the aneurysm, as well as a mechanism to seal off the aneurysmal sac from the blood flow by sealingly engaging the blood vessel wall at the opposed ends thereof. The graft may be manufactured in sheet or tubular form, such as by weaving, knitting or braiding the graft material into a fabric sheet or tube. The stent provides rigidity and structure, to hold the graft open in the tubular shape, as well as to press the graft material into engagement with the blood vessel wall to effectuate the sealing therewith. The stent is typically manufactured by folding or bending individual elements of wire, laser or other cutting of sheets or tubes, or otherwise forming shapes to provide a relatively rigid structure to support the graft.

In one embodiment, to attach the graft to the stent, the graft is typically inserted into, or pulled over, the stent, and the graft is sewn to the structural components of the stent. Alternatively, the stent may be formed on the graft such that the individual wires of the stent are threaded though specially provided projecting fabric loops on the surface of the graft, thereby creating attachment of the graft to the stent. The stent and graft are sized such that upon placement thereof into an aneurysmal blood vessel, the diameter of the stent graft slightly exceeds the existing diameter of the blood vessel at healthy blood vessel wall site adjacent to the aneurysm.

In another embodiment, an exclusion device has a stent graft structure, wherein the stent and graft are integrally formed such that the stent and graft are formed as a single unitary body. In one aspect, the stent graft includes a graft, formed of a fluid barrier material, within which is formed a stent material, as an integral part thereof In one embodiment, the stent graft is woven, such that a graft material, formed of fibers, is integrally woven with a stent material, so that a resulting stent graft is formed having the stent integrally provided with the graft. In another embodiment, the stent and graft materials are interbraided, such that individual filaments of the ultimate stent structure are integrally braided with the material forming the graft, such that an integral stent graft is formed.

In conventional designs of stent grafts, the ends of the stent are cut substantially perpendicular to the long axis of the stent Therefore, conventional designs of stent grafts suffer from the same concerns expressed above for conventional stents.

It is therefore a need to provide stent designs that facilitate the current practices of cannulation.

There is also a need to provide stent designs that do not result on narrowing (bottle necking) of the mouth of the branch arteries.

There is a further need to provide stent designs that accommodate the curvature of tortuous arteries.

### BRIEF SUMMARY

In one embodiment, the stent of these teachings includes a substantially cylindrical expandable structure having two ends, a locus of points at one of the two ends defining a surface, the surface being beveled with respect to a central axis of the substantially cylindrical expandable structure.

The beveled end provides a number of benefits. In one instance, the angle of the bevel on the proximal end of the stent improves the accessibility of the stented artery for catheters for subsequent endovascular procedures. That is, the angle of the stent allows a catheter to be more easily inserted into the lumen of the artery without being obstructed by the proximal end of the stent or becoming entrapped between the outer surface of the stent and the inner surface of the artery. In another instance, when stenting a branch artery, the beveled end allows the stent to be placed such that the beveled end avoids narrowing (bottle necking) of the proximal end of the branch artery and, therefore, achieves better blood flow to the branch artery. In a further instance, the beveled end prevents the presence of the stent from exaggerating or exasperating the twists of a tortuous (twisted) artery at the distal end of the stent and, therefore, achieving better blood flow at the site.

The method pertains to the deployment of the new stent to achieve a number of benefits. These benefits include, but are not limited to, placement of the stent to: (1) improve the accessibility of the stented artery for catheters for subsequent endovascular procedures, (2) achieve better blood flow to the branch artery by avoiding narrowing (bottlenecking) of the mouth of the branch artery, and (3) achieve better blood flow in a tortuous artery by preventing the presence of the stent from exaggerating or exasperating the twists of the vessel at the distal end of the stent.

For a better understanding of the present teachings, together with other and further needs thereof, reference is made to the accompanying drawings and detailed description and its scope will be pointed out in the appended claims.

### BRIEF DESCRIPTION OF THE DRAWINGS

FIG. 1 illustrates a stent incorporating features of the teachings which is mounted onto a delivery catheter;

FIG. 2 shows an embodiment of the stent of these teachings holding open an artery;

FIG. 3 depicts cannulation with a conventional stent;

FIG. 4 depicts“bottle necking” due to a conventional stent;

FIG. 5 depicts a conventional stent used in a tortuous artery;

FIG. 6 depicts an application of an embodiment of the stent of these teachings;

FIG. 7 depicts another application of an embodiment of the stent of these teachings;

FIG. 8 depicts an application of an embodiment of the stent of these teachings; and

FIG. 9 shows an embodiment of a multi-stage stent graft of these teachings.

### DETAILED DESCRIPTION

In one embodiment, the stent of these teachings includes a substantially cylindrical expandable structure having two ends, a locus of points at one of the two ends defining a surface, the surface being beveled with respect to a central axis of the substantially cylindrical expandable structure.

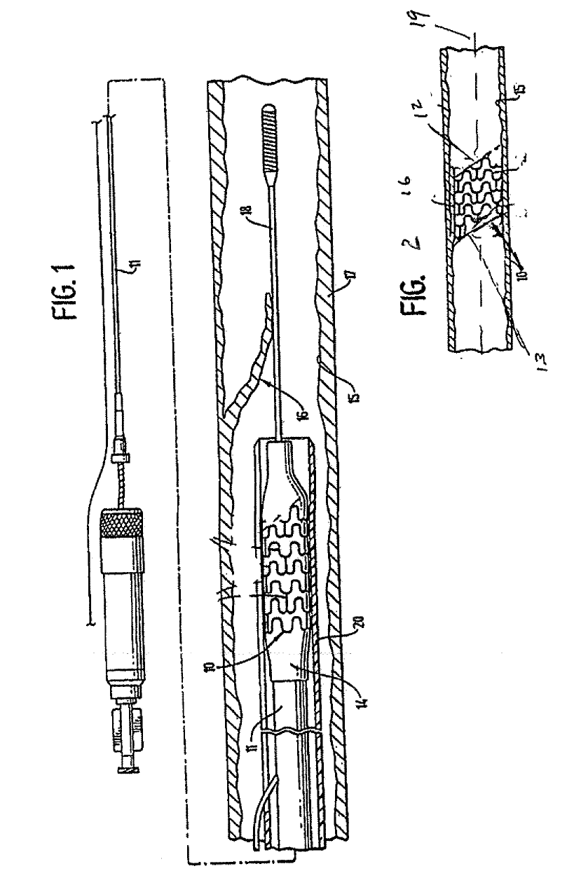

FIG. 1 illustrates a stent 10 incorporating features of the teachings which is mounted onto a delivery catheter 11. In one instance, such as that described in U.S. Pat No. 6,432,133, which is incorporated by reference herein, the stent of these teachings not being limited to this instance, the stent generally comprises a plurality of radially expandable cylindrical interconnected elements. The delivery catheter 11 has an expandable portion or balloon 14 for expanding of the stent 10 within an artery 15. The artery 15, as shown in FIG. 1 has a dissected lining 16 which has occluded a portion of the arterial passageway.

The delivery catheter 11 onto which the stent 10 is mounted, is essentially the same as a conventional balloon dilatation catheter for angioplasty procedures. The balloon 14 may be formed of suitable materials such as, but not limited to, polyethylene, polyethylene terephthalate, polyvinyl chloride, nylon and ionomers such as Surlyn™ manufactured by the Polymer Products Division of the Du Point Company. Other polymers may also be used. In order for the stent 10 to remain in place on the balloon 14 during delivery to the site of the damage within the artery 15, the stent 10 is compressed onto the balloon. One embodiment of a retractable protective delivery sleeve 20, such as, but not limited to, that described in U.S. Pat. No. 5,507,768, entitled STENT DELIVERY SYSTEM, which is incorporated by reference herein, may be provided to further ensure that the stent stays in place on the expandable portion of the delivery catheter 11 and prevent abrasion of the body lumen by the open surface of the stent 20 during delivery to the desired arterial location. Other means for securing the stent 10 onto the balloon 14 may also be used, such as providing collars or ridges on the ends of the working portion, i.e. the cylindrical portion, of the balloon.

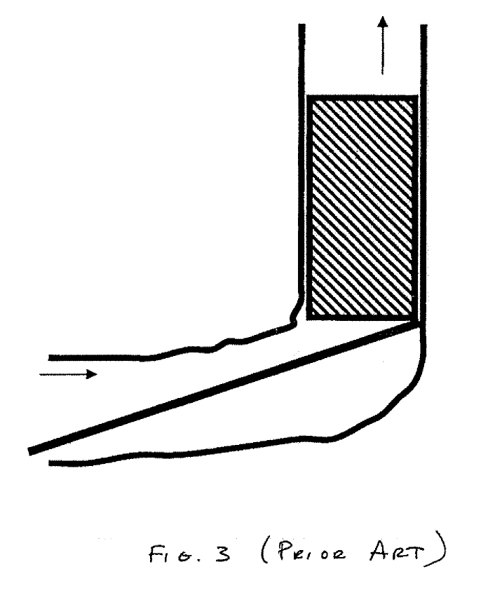

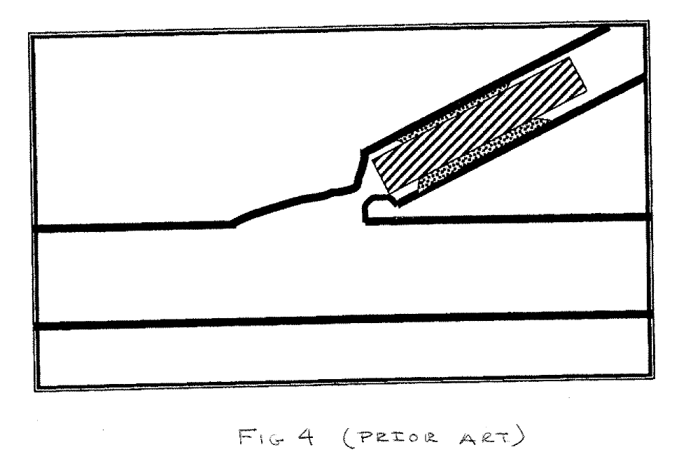

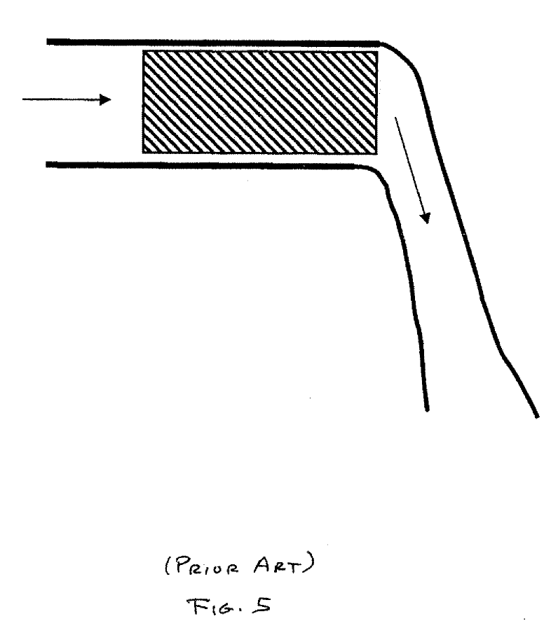

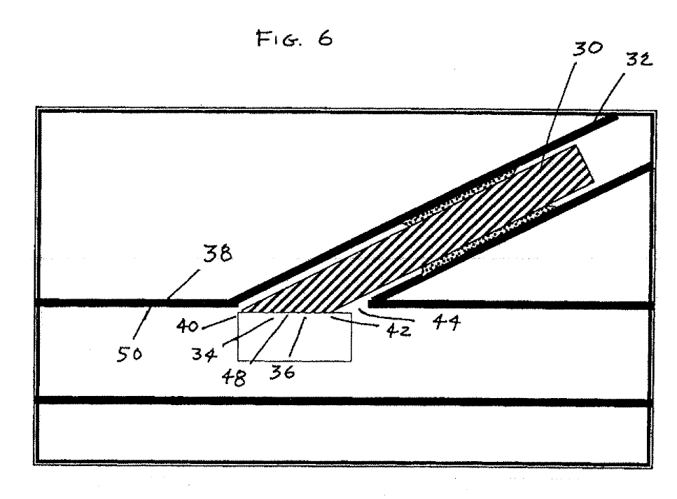

FIG. 2 shows an embodiment of the stent of these teachings 10 holding open the artery 15 after the catheter 11 is withdrawn. Referring to FIG. 2, the stent 10 is a substantially cylindrical expandable structure having two ends 12, 13. A locus of points at one of the two ends 12, 13 defines a surface, which is beveled with respect to a central axis 19 of the stent 10. In the embodiment shown in FIG. 2, both ends 12, 13 define surfaces that are beveled. In the instance in which both surfaces are beveled, embodiments in which an angle between a normal to the first beveled surface and the central axis is different from an angle between a normal to the other beveled surface and the central axis.

The stent of these teachings may be constructed of any material that permits the structure to be expandable, rigid upon expansion, and that is compatible with the use as a stent in a vessel of the human body.

In one embodiment, the stent of these teachings is fabricated of a shape memory alloy, which is encapsulated in its final diametric dimension, and the encapsulated intraluminal stent-graft is manipulated into its reduced diametric dimension and radially expanded in vivo under the influence of a transformation.

The term “shape memory” is used in the art to describe the property of a material to recover a pre-programmed shape after deformation of a shape memory alloy in its martensitic phase and exposing the alloy to a temperature excursion through its austenite transformation temperature, at which temperature the alloy begins to revert to the austenite phase and recover its preprogrammed shape. The term “pseudoelasticity” is used to describe a property of shape memory alloys where the alloy is stressed at a temperature above the transformation temperature of the alloy and stress-induced martensite is formed above the normal martensite formation temperature. Because it has been formed above its normal temperature, stress-induced martensite reverts immediately to undeformed austenite as soon as the stress is removed, provided the temperature remains above the transformation temperature.

Shape memory alloys are a group of metallic materials that demonstrate the ability to return to a defined shape or size when subjected to certain thermal or stress conditions. Shape memory alloys are generally capable of being plastically deformed at a relatively low temperature and, upon exposure to a relatively higher temperature, return to the defined shape or size prior to the deformation. Shape memory alloys may be further defined as one that yields a thermoplastic martensite. A shape memory alloy which yields a thermoplastic martensite undergoes a martensitic transformation of a type that permits the alloy to be deformed by a twinning mechanism below the martensitic transformation temperature. The deformation is then reversed when the twinned structure reverts upon heating to the parent austenite phase. The austenite phase occurs when the material is at a low strain state and occurs at a given temperature. The martensite phase may be either temperature-induced martensite (TIM) or stress-induced martensite (SIM).

In one embodiment, the stent of these teachings utilizes a binary, equiatomic nickel-titanium alloy because of its biocompatibility and because such an alloy exhibits a transformation temperature within the range of physiologically-compatible temperatures. The dimensions of the stent of these teachings are chosen to accommodate placement within vascular vessels.

The stent of these teachings includes a structural lattice such as, but not limited to, the lattice disclosed in U.S. Pat. No. 6,432,133 and in U.S. Pat. No. 5,354,308, both of which are incorporated by reference herein.

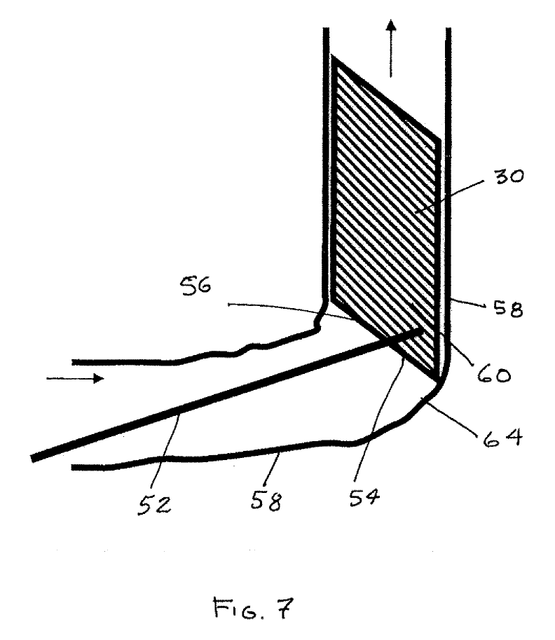

The stent of these teachings allows branch arteries to be stented in a way that reduces bottlenecking at the mouth of the branch artery, as shown in FIG. 6. An embodiment of the stent 30 of these teachings is inserted into the branch artery 32 such that only the bevel 34 of the proximal end 36 extends into the main artery 38. The bevel 34 is oriented such that the short end 40 of the bevel is flush with the mouth 44 of the branch artery 32 and the long end 42 of the bevel extends slightly into the main artery 38 (this is tantamount to orienting the beveled end 34 so that the beveled surface 48 is substantially aligned with the surface 50 of the main artery 38 in a vicinity of the mouth 44 of the branch artery 32). The short end 40 of the bevel should align with the upstream side of the mouth 44 of the branch artery 32 and long end 42 of the bevel should protrude from the downstream side of the mouth 44 of the branch artery 32. This orientation allows the stent 30 to provide structural support to the full circumference of the mouth 44 of the branch artery 32 and prevents the mouth 44 of the branch artery 32 from narrowing.

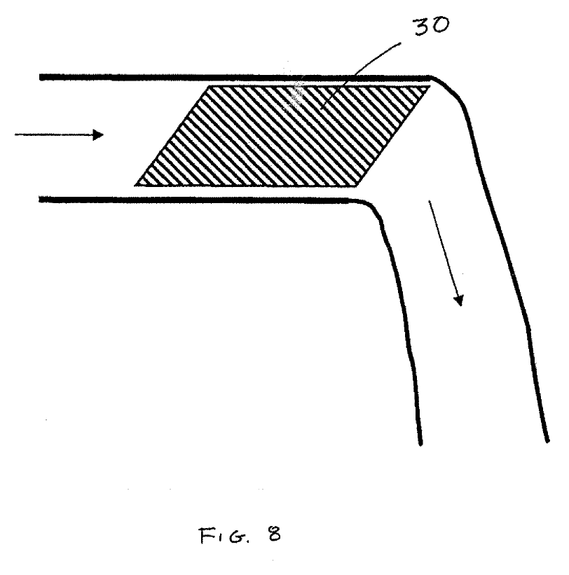

As shown in FIG. 7, the stent of these teachings 30 allows an angled catheter 52 to be placed into the proximal end 54 of the stent 30 with less difficulty than stents that feature perpendicular ends. The diagonal cut of the beveled end 56 of stent of these teachings 30 provides a more gradual transition between the vessel 58 and the stent 30. This beveled end 56 allows a catheter 52 to be inserted into the lumen 60 of the stent 30 and artery 58 without colliding with the proximal end 54 of the stent 30 or becoming entrapped between the outer surface of the stent 30 and the inner surface 64 of the artery 58.

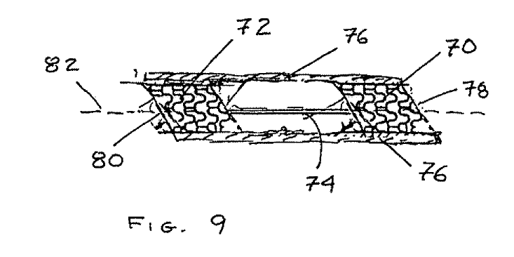

As shown in FIG. 8, the stent of these teachings 30 also allows allows the stent 30 to conform to the natural curvature of a tortuous artery. This minimizes further kinking of the artery at the distal end of the stent and contributes toward proper blood flow at the site.

In one embodiment, the stent of these teachings, when covered with a synthetic material or when having structural elements, comprising synthetic material, interdigitated therein, is a stent graft. In one embodiment, the synthetic material is selected from the group consisting of polyester, polytetrafluoroethylene, microporous urethane, nylon, and lycra. The stent graft of these teachings can have structural elements interdigitated therein as described in U.S. patent application Ser. No. 10/423,370, also published as U.S. Patent Application Publication No. US 2004/0215320 A1, both of which are incorporated by reference herein.

In another embodiment, the stent graft of these teachings includes a multi-stage stent graft as described in U.S. Pat. Nos. 6,280,467 and 6,572,645, both of which are incorporated by reference herein. The multi-stage stent graft of these teachings, shown in FIG. 9, includes a first support stent 70, a second support stent 72, the first support stent 70 being spaced apart from the second support stent 72 and connected to the second support stent by at least one connecting element 74. A tubular graft 76 comprising a graft material formed into a tubular shape is disposed to surround a portion of both the first support stent 70 and the second support stent 72. In at least one of the support stents, a locus of points at the end that is not opposite to the other support stent defines a surface 78, 80, the surface being beveled with respect to a central axis 82 of the support stents (and the tubular graft).

The synthetic material for the tubular graft 76 is pliable enough to substantially conform to the interior surface of a blood vessel being treated. Suitable synthetic materials include, but are not limited to, woven polyester, polytetratluoroethylene (PTFE), microporous urethane, nylon and lycra. A preferred fabric material is polyester.

In some embodiments of the present teachings, a photopolymerization technique is used to treat the synthetic material of the tubular graft 76. While not intending to be bound by any particular theory, it are believed that photopolymerization makes the surface of the synthetic material conducive to bonding of proteins which are necessary to create a collagen rich surface thereon. This enables a thinner, higher porosity fabric to be utilized without bleed-through and also promotes healing. Further, selection of the synthetic graft material depends upon the site of implantation. For example, polyester (Dacron) is preferred for the aortic wall which experiences a higher pressure change than for example, the iliac artery where, PTFE is the preferred material.

Although the teachings have been described with respect to various embodiments, it should be realized these teachings are also capable of a wide variety of further and other embodiments within the spirit and scope of the appended claims.

What is claimed is:

1. A stent comprising:
a substantially cylindrical expandable structure having two ends; a locus of points at one of said two ends defining a surface, said surface being beveled with respect to a central axis of said substantially cylindrical expandable structure.

2. The stent of claim 1 wherein a locus of points at another one of said two ends defines another surface, said another surface being beveled with respect to the central axis of said substantially cylindrical expandable structure.

3. The stent of claim 2 wherein an angle between a normal to said surface and said central axis is different from an angle between a normal to said another surface and said central axis.

4. The stent of claim 1 wherein said substantially cylindrical expandable structure comprises a plurality of interconnected elements.

5. The stent of claim 1 wherein said substantially cylindrical expandable structure is covered with a synthetic material.

6. The stent of claim one wherein said substantially cylindrical expandable structure has structural elements interdigitated therein.

7. A stent graft comprising:
a first support stent;
a second support stent;
said first support stent being spaced apart from said second support stent and connected to said second support stent by at least one connecting element;
said first support stent having two ends, said two ends comprising one end closest to said second support stent and another end; a locus of points at said another end defining a surface, said surface being beveled with respect to a central axis of said first support stent; and
a tubular graft comprising a graft material formed into a tubular shape, wherein said tubular graft is disposed to surround a portion of both said first support stent and said second support stent.

8. The stent graft of claim 7 wherein said second support stent has two ends, said two ends comprising one end closest to said first support stent and another end; a locus of points at said another end defining a surface, said surface being beveled with respect to a central axis of said second support stent.

9. The stent graft of claim 7 wherein said graft material is a synthetic material.

10. The stent graft of claim 9 wherein said synthetic material selected from the group consisting of polyester, polytetrafluoroethylene, microporous urethane, nylon, and lycra.

11. A method for stenting a branch artery, the method comprising the steps of:
providing a stent having a proximal end, a locus of points at the proximal end defining a surface, the surface being beveled with respect to a central axis of the stent, the proximal end comprising a beveled end;
inserting the stent into the branch artery, the beveled end being located at a mouth of the branch artery; and
orienting the beveled end, the surface being substantially aligned with the surface of a main artery in a vicinity of the mouth of the branch artery.

12. A method for placing a catheter into a lumen of a stented vessel, the method comprising the step of:
providing a stent having a proximal end, a locus of points at the proximal end defining a surface, the surface being beveled with respect to a central axis of the stent; and
inserting the catheter, through the proximal end, into a lumen of the stent.
